(19) United States
(12) Patent Application Publication
(10) Pub. No.: US 2009/0162420 A1
(43) Pub. Date: Jun. 25, 2009

(54) Matrix-Controlled Transdermal System Comprising Salts of ACE Inhibitor Dicarboxylic Acids

(76) Inventors: Karin Klokkers, Reicfusbeveun (DE); Michael Helfrich, München (DE); Jörg Nink, München (DE)

Correspondence Address: LICATA & TYRRELL P.C., 66 E. MAIN STREET, MARLTON, NJ, 08053, US

(21) Appl. No.: 12/096,078

(22) PCT Filed: Dec. 5, 2006
(86) PCT No.: PCT/EP2006/011672

§ 371 Date: Nov. 12, 2008

(30) Foreign Application Priority Data
Dec. 5, 2005 (DE) 10 2005 058 166.8

(51) Int. Cl. A61K 9/70; C07D 207/24; C07D 233/36; C07F 9/572; C07D 217/04; C07D 209/42; C07D 495/10; C07D 223/16; A61K 31/19; A61K 31/55; A61K 31/551; A61K 31/403; A61K 31/4164; A61K 31/675; A61K 31/472; A61K 31/407

(52) U.S. Cl. 424/443; 562/400; 548/541; 548/322.5; 548/413; 546/147; 548/492; 548/409; 540/523; 514/557; 514/212.07; 514/221; 514/412; 514/392; 514/91; 514/307; 514/409

(57) ABSTRACT

The invention relates to a salt of an ACE inhibitor dicarboxylic acid with an organic amine and/or an alkali compound, a transdermal therapeutic system comprising the salt, and a method of producing the transdermal therapeutic system.

The invention relates to a stable, active ingredient-containing transdermal therapeutic system for the administration of ACE inhibitors, the metabolites of which constitute a dicarboxylic acid. Stable neutral derivatives of ACE inhibitor dicarboxylic acids are obtained by salt formation with an organic amine and/or with an alkali compound. Preferably, stable, neutral derivatives of ACE inhibitor dicarboxylic acids are obtained with an organic amine, and especially preferably with a molar equivalent of an organic amine.

Long-term therapy of hypertonia with angiotensin-converting enzyme inhibitors (ACE inhibitors) is becoming increasingly widespread.

ACE inhibitors are known for their reliable activity while at the same time being well tolerated. Only injectable forms or oral administration forms of ACE inhibitors, such as tablets or capsules, have so far been available on the market. A disadvantage of the use of injectable forms is low patient compliance. Oral forms of administration have the disadvantage that the patient has to swallow at least one tablet or capsule each day and the blood plasma level is always subject to certain variations. A constant plasma level can scarcely be guaranteed with oral forms of administration.

Transdermal application, on the other hand, offers a number of advantages for ACE inhibitors:

access to the skin is not limited,
there is no change of environment, as in the case of peroral administration,
handling is simple and convenient,
normally, a single administration is sufficient for at least 3 days, instead of multiple daily administrations,
patient compliance is substantially improved,
continuous long-term therapy is possible,
the release of the active ingredient is approximately in accordance with zero order kinetics,
therapy can be discontinued more quickly,
a constant plasma level is ensured over a relatively long period,
a plasma level that is initially too high, as in the case of intravenous administration, is avoided, and
because of the avoidance of first pass, in some cases a lower dose is required than for oral administration, as a result of which there is a lower side-effect rate, and the risk of overdose or underdose is lower.

EP 0 439 430 describes a transdermal reservoir system having an ACE inhibitor content, which system contains a) a top layer impermeable to active ingredient (closed outer layer), b) an active ingredient-containing reservoir comprising a carrier or solvent and also, optionally, a membrane, c) an adhesive layer and d) a removable protective layer (peel-off protective layer). A salt of a zwitterion serves as the active ingredient, wherein there may be provided as zwitterion, for example, benazeprilate and, as salt-forming component, for example, chlorprocaine, choline, diethanolamine, ethylenediamine, methylglucamine, procaine or an alkali metal hydroxide, carbonate or hydrogen carbonate, for example of lithium or potassium, and examples of di-salts that are mentioned are dilithium benazeprilate and dipotassium benazeprilate (Table).

EP 0 452 837 describes a matrix for plasters which, inter alia, contains ACE inhibitors as active ingredients. As possible ACE inhibitors there are mentioned delapril hydrochloride, enalapril maleate, captopril, alacepril and (R)-3-[(S)-1-carboxy-5-(4-piperidyl)-pentyl]-amino-4-oxo-2,3,4,5-tetrahydro-1,5-benzothiapin-5-acetic acid (=dicarboxylic acid). Basic substances are used therein as solubilisers only in a very small amount.

WO 96/29 999 discloses a TTS having a matrix based on polyisobutylene or butyl rubber with a content of trandolapril and/or ramipril.

Described in WO 02/03 970 is a matrix-TTS in which the matrix layer contains an ACE inhibitor in the form of a dicarboxylic acid that has been converted to a derivative from the following group: diesters, di-salts obtainable with bases and mono-salts obtainable with acids. A TTS containing a di-salt of a base, however, has been shown to exhibit a high level or skin irritation.

ACE inhibitors frequently exhibit a low degree of stability and may undergo various decomposition reactions. ACE inhibitor dicarboxylic acids having the structural element below may, for example, convert to substituted diketopiperazines. By way of nucleophilic attack of the nitrogen atom, intramolecular lactam formation occurs, as shown by the following equation:

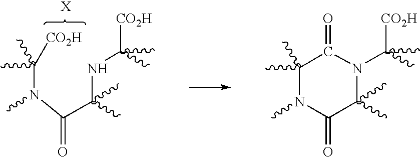

The intramolecular lactam formation can be prevented by the addition of acids which are able to block the secondary amine group. The salts of the ACE inhibitor dicarboxylic acids formed with acids in that way (see WO 02/03790) have the disadvantage that there is an increased potential for skin irritation to occur as a result of the low pH value.

When di-salts of the ACE inhibitor dicarboxylic acids with bases (see WO 02/03790) are used, intramolecular lactam formation is likewise prevented, but the formulation as a whole has a basic pH value, which can likewise result in skin irritation.

The problem of the present invention is to provide a matrix-TTS having a content of salts of ACE inhibitor dicarboxylic acids, with a high degree of stability and a low skin irritation potential. The active ingredient content is to be stable over a relatively long time period and to be subject to as little as possible in the way of decomposition processes. The transdermal system is to exhibit a high flux, that is, the active ingredient is to be discharged with a high skin-permeation rate.

The problem underlying the invention is solved in that a salt, preferably a mono-salt, of an ACE inhibitor dicarboxylic acid is prepared with an organic amine and/or with an alkali compound.

The salt according to the invention may be prepared with a monoamine as organic amine.

The salt according to the invention may accordingly be prepared with a primary amine, a secondary amine or a tertiary amine as organic amine.

The salt according to the invention may accordingly be prepared with an aliphatic primary C₄₋₁₂amine.

The salt according to the invention may accordingly be prepared with butylamine, pentylamine, hexylamine, heptylamine, octylamine, nonylamine, decylamine, undecylamine, dodecylamine or trometamol (=2-amino-2-hydroxymethyl-1,3-propanediol) as aliphatic primary C₄₋₁₂amine.

Also, the salt according to the invention may be prepared with pyrrolidone or a derivative thereof as secondary amine.

Also, the salt according to the invention may be prepared with triethanolamine as tertiary amine.

The salt according to the invention may be prepared with an alkali compound, an alkali compound in the context of this compound being any compound that includes an alkali metal cation. Salts of the ACE inhibitor dicarboxylic acid are accordingly preferably formed with an alkali metal cation, and are also called alkali salts of the ACE inhibitor dicarboxylic acid. Examples of suitable alkali metal cations are lithium, sodium, potassium, caesium and rubidium cations, of which lithium, sodium and potassium cations are especially preferred. Also preferred as a cation is ammonium (NH₄⁺) (pseudo-alkali metal cation). Preferably, the counter anion of the alkali compound, especially of the alkali metal cation, has proton acceptor properties, and the alkali compound is especially preferably an alkali metal cation-containing salt of a strong or weak, inorganic or organic acid. Preferred examples are alkali compounds such as alkali metal hydroxides, such as, for example, lithium hydroxide, sodium hydroxide or potassium hydroxide, alkali metal alcoholates, such as, for example, lithium methanolate, lithium ethanolate, sodium methanolate, sodium ethanolate, potassium methanolate or potassium ethanolate, or alkali metal carbonates, such as, for example, sodium carbonate or potassium carbonate, or alkali metal hydrogen carbonates, such as, for example, sodium hydrogen carbonate or potassium hydrogen carbonate, or alkali metal tartrates, such as, for example, sodium tartrate or potassium tartrate, or alkali metal maleates, such as, for example, sodium maleate or potassium maleate. Ammonium hydroxide is preferred as ammonium compound.

The salt according to the invention may be prepared with an alkaline earth compound, an alkaline earth compound in the context of this compound being any compound that includes an alkaline earth metal cation. Salts of the ACE inhibitor dicarboxylic acid are accordingly preferably formed with an alkaline earth metal cation, and are also called alkaline earth salts of the ACE inhibitor dicarboxylic acid. Examples of suitable alkaline earth metal cations are magnesium cations or calcium cations. Preferably, the counter anion of the alkaline earth compound, especially of the alkaline earth metal cation, has proton acceptor properties, and the alkaline earth compound is especially preferably an alkaline earth metal cation-containing salt of a strong or weak, inorganic or organic acid. Preferred examples are alkaline earth compounds such as alkaline earth metal hydroxides, such as, for example, magnesium hydroxide or calcium hydroxide, or alkaline earth metal carbonates, such as, for example, magnesium carbonate or calcium carbonate. In the case of a salt according to the invention with an alkaline earth compound, the molar ratio of ACE inhibitor dicarboxylic acid alkaline earth compound is preferably from 1:0.5 to 1: less than 1, further preferably from 1:0.5 to 1:0.9, especially from 1:0.5 to 1:0.55.

The salt according to the invention encompasses a salt with one or more organic amine(s), preferably one, two or three organic amine(s), or with one or more alkali compound(s), preferably one, two or three alkali compound(s), or with one or more alkaline earth compound(s), preferably one, two or three alkaline earth compound(s), and any desired mixtures thereof. Preferably, the salt according to the invention encompasses a salt with two organic amines that are different from each other, or two alkali compounds that are different from each other, or two alkaline earth compounds that are different from each other, or one organic amine and one alkali compound, and the like.

The salt according to the invention may be a salt of an ACE inhibitor dicarboxylic acid from the group of the dicarboxylic acids of imidapril, fosinopril, moexipril, perindopril, spirapril, benazepril, cilazapril, lisinopril, quinapril, enalapril, delapril, ramipril and trandolapril.

A salt according to the invention of an ACE inhibitor dicarboxylic acid of trandolapril or ramipril may accordingly be considered.

The molar ratio of ACE inhibitor dicarboxylic acid: amine or ACE inhibitor dicarboxylic acid alkali compound for the salt according to the invention may be from 1: less than 2.

Accordingly, the molar ratio of ACE inhibitor dicarboxylic acid: amine or ACE inhibitor dicarboxylic acid: alkali compound for the salt according to the invention may be from 1:0.5 to 1: less than 2, preferably from 1:0.5 to 1:1.9, further preferably from 1:0.9 to 1:1.5, further preferably of 1: from 1 to 1.5, especially preferably of 1:1.1 and especially of approximately 1:1.

The problem underlying the invention is furthermore solved by a transdermal therapeutic system comprising as active ingredient at least one salt of an ACE inhibitor dicarboxylic acid with at least one organic amine and/or at least one alkali compound. Especially preferably, the problem underlying the invention is solved by a transdermal therapeutic system comprising as active ingredient at least one mono-salt of an ACE inhibitor dicarboxylic acid with at least one organic amine and/or at least one alkali compound, especially comprising as active ingredient a mono-salt of an ACE inhibitor dicarboxylic acid with an organic amine.

A monoamine may be provided as organic amine for the transdermal therapeutic system according to the invention.

A primary amine, a secondary amine or a tertiary amine may accordingly be provided as organic amine for the transdermal therapeutic system according to the invention.

An aliphatic primary C₄₋₁₂amine may accordingly be provided for the transdermal therapeutic system according to the invention.

Butylamine, pentylamine, hexylamine, heptylamine, octylamine, nonylamine, decylamine, undecylamine, dodecylamine, or trometamol (=2-amino-2-hydroxymethyl-1,3-propanediol) may accordingly be provided as aliphatic primary C₄₋₁₂amine for the transdermal therapeutic system according to the invention.

Further, the transdermal therapeutic system according to the invention may be prepared with pyrrolidone or a derivative thereof as secondary amine.

Further, the transdermal therapeutic system according to the invention may be prepared with triethanolamine as tertiary amine.

Further, the transdermal therapeutic system according to the invention may be prepared with an alkali compound, an alkali compound in the context of this compound being any compound that includes an alkali metal cation. Examples of suitable alkali metal cations are lithium, sodium, potassium, caesium and rubidium cations, of which lithium, sodium and potassium cations are especially preferred. Also preferred as a cation is ammonium (NH₄⁺) (pseudo-alkali metal cation). Preferably, the counter anion of the alkali compound, especially of the alkali metal cation, has proton acceptor properties, and the alkali compound is especially preferably an alkali metal cation-containing salt of a strong or weak, inorganic or organic acid. Preferred examples are alkali compounds such as alkali metal hydroxides, such as, for example, lithium hydroxide, sodium hydroxide or potassium hydroxide, alkali metal alcoholates, such as, for example, lithium methanolate, lithium ethanolate, sodium methanolate, sodium ethanolate, potassium methanolate or potassium ethanolate, or alkali metal carbonates, such as, for example, sodium carbonate or potassium carbonate, or alkali metal hydrogen carbonates, such as, for example, sodium hydrogen carbonate or potassium hydrogen carbonate, or alkali metal tartrates, such as, for example, sodium tartrate or potassium tartrate, or alkali metal maleates, such as, for example, sodium maleate or potassium maleate. Ammonium hydroxide is preferred as ammonium compound.

Further, the transdermal therapeutic system according to the invention may be prepared with an alkaline earth compound, an alkaline earth compound in the context of this compound being any compound that includes an alkaline earth metal cation. Examples of suitable alkaline earth metal cations are magnesium or calcium cations. Preferably, the counter anion of the alkaline earth compound, especially of the alkaline earth metal cation, has proton acceptor properties, and the alkaline earth compound is especially preferably an alkaline earth metal cation-containing salt of a strong or weak, inorganic or organic acid. Preferred examples are alkaline earth compounds such as alkaline earth metal hydroxides, such as, for example, magnesium hydroxide or calcium hydroxide, or alkaline earth metal carbonates, such as, for example, magnesium carbonate or calcium carbonate. In the case of a salt according to the invention with an alkaline earth compound, the molar ratio of ACE inhibitor dicarboxylic acid: alkaline earth compound is preferably from 1:0.5 to 1: less than 1, further preferably from 1:0.5 to 1:0.9, especially from 1:0.5 to 1:0.55.

Further, the transdermal therapeutic system according to the invention may be prepared with one or more organic amine(s), preferably one, two or three organic amine(s), or with one or more alkali compound(s), preferably one, two or three alkali compound(s), or with one or more alkaline earth compound(s), preferably one, two or three alkaline earth compound(s), and any desired mixtures thereof. Preferably, the salt according to the invention encompasses a salt with two organic amines that are different from each other, or two alkali compounds that are different from each other, or two alkaline earth compounds that are different from each other, or one organic amine and one alkali compound, and the like.

Further, the transdermal therapeutic system according to the invention may be provided with at least one salt of an ACE inhibitor dicarboxylic acid from the group of the dicarboxylic acids of imidapril, fosinopril, moexipril, perindopril, spirapril, benazepril, cilazapril, lisinopril, quinapril, enalapril, delapril, ramipril and trandolapril.

Preferably, the transdermal therapeutic system according to the invention may be prepared with at least one mono-salt of an ACE inhibitor dicarboxylic acid from the group of the dicarboxylic acids of imidapril, fosinopril, moexipril, perindopril, spirapril, benazepril, cilazapril, lisinopril, quinapril, enalapril, delapril, ramipril and trandolapril, or mixtures of two, three or more thereof.

The transdermal therapeutic system according to the invention may accordingly be prepared with a salt of an ACE inhibitor dicarboxylic acid of trandolapril and/or ramipril.

Preferably, the transdermal therapeutic system according to the invention may be prepared with a mono-salt of an ACE inhibitor dicarboxylic acid of trandolapril and/or ramipril.

The molar ratio of ACE inhibitor dicarboxylic acid: amine or ACE inhibitor dicarboxylic acid alkali compound for the transdermal therapeutic system according to the invention may be from 1: less than 2.

Accordingly, the molar ratio of ACE inhibitor dicarboxylic acid: amine or ACE inhibitor dicarboxylic acid: alkali compound for the transdermal therapeutic system according to the invention may be from 1:0.5 to 1: less than 2, preferably from 1:0.5 to 1:1.9, further preferably from 1:0.9 to 1:1.5, further preferably of 1: from 1 to 1.5, especially preferably of 1:1.1 and especially of approximately 1:1.

The transdermal therapeutic system according to the invention may be provided with
a top layer impermeable to active ingredient,
one or more active ingredient-containing self-adhesive matrix layers and
a peel-off protective layer.

Further, the transdermal therapeutic system according to the invention may be provided with
a top layer impermeable to active ingredient,
one or more active ingredient-containing matrix layers,
which is(are) provided on the application side with a layer of contact adhesive, and
a peel-off protective layer.

The transdermal therapeutic system according to the invention may accordingly be provided with a non-self-adhesive matrix layer and a separate layer of contact adhesive.

For the transdermal therapeutic system according to the invention, the active ingredient or active ingredients, that is to say, one or more salts, especially one or more mono-salts, of an ACE inhibitor dicarboxylic acid with at least one organic amine and/or at least one alkali compound, may be dissolved and/or be present in the form of droplets of emulsion in the matrix.

The content of ACE inhibitor dicarboxylic acid for the transdermal therapeutic system according to the invention may be from 2 to 35% by weight, based on the weight of the matrix.

The content of ACE inhibitor dicarboxylic acid for the transdermal therapeutic system according to the invention may accordingly be from 10 to 25% by weight, based on the weight of the matrix.

The transdermal therapeutic system according to the invention may be prepared with a pressure-sensitive adhesive based on polyurethane, polyisobutylene, polyvinyl ether, polyacrylate, silicone, styrene block copolymer or a mixture thereof.

The transdermal therapeutic system according to the invention may accordingly be prepared with a pressure-sensitive adhesive based on styrene-isoprene-styrene block copolymer (SIS) or styrene-butadiene-styrene block copolymer.

Also, the transdermal therapeutic system according to the invention may be prepared with a pressure-sensitive adhesive based on polyacrylate or polyisobutylene.

Further, the transdermal therapeutic system according to the invention may be prepared with a matrix former from the group of polyacrylate, polyisobutylene, silicone, styrene block copolymer or a mixture thereof.

The transdermal therapeutic system according to the invention may accordingly be prepared with a styrene-isoprene-styrene block copolymer (SIS) as matrix former.

Also, the transdermal therapeutic system according to the invention may be prepared with a self-adhesive matrix based on polyacrylate.

Further, the transdermal therapeutic system according to the invention may be prepared with a contact adhesive and/or a matrix based on polyacrylate, which may be a homopolymer, copolymer or terpolymer.

The transdermal therapeutic system according to the invention may accordingly be prepared with a contact adhesive and/or a matrix based on polyacrylate, containing or consisting of various acrylic acid derivatives.

The transdermal therapeutic system according to the invention may accordingly be prepared with a contact adhesive and/or a matrix based on polyacrylate, consisting of acrylate polymer of
at least 50% by weight of an acrylate, methacrylate, alkyl acrylate, alkyl methacrylate or acrylamide monomer,
from 0 to 20% by weight of a functional monomer, copolymerisable with acrylate, and
from 0 to 50% by weight of another monomer.

Further, the transdermal therapeutic system according to the invention may be prepared with a permeation enhancer from the group formed by

saturated and/or unsaturated fatty alcohols each having from 8 to 18 C atoms; tea tree oil;
saturated and/or unsaturated cyclic ketones;
alkyl methyl sulphoxides;
saturated and/or unsaturated fatty acids each having from 8 to 18 C atoms;
esters of saturated and/or unsaturated fatty acids each having from 8 to 18 C atoms;
salts of saturated and/or unsaturated fatty acids each having from 8 to 18 C atoms;
natural vitamin E;
synthetic vitamin E and/or vitamin E derivatives;
sorbitan fatty acid esters;
ethoxylated sorbitan fatty acid esters;
azones, especially laurocapram;
1-alkylpyrrolidone;
block copolymers of polyethylene glycol and dimethylsiloxane having a cationic group at one end;
polyoxyethylene-10 stearyl ether;
a mixture of polyoxyethylene-10 stearyl ether and glyceryl dilaurate;
dodecyl-2-(N,N-dimethylamino)-propanoltetradecanoate and/or dodecyl 2-(N,N-dimethylamino)-propionate;
N-acetylprolinate esters (N-acetyl-pyrrolidone-2-carboxylic acid esters) having >8 C atoms; non-ionic surfactants, especially lauryl ether;
esters of polyoxyethylene;
dimethyl(arylimino)sulphuran;
a mixture of oleic acid analogue(s) and propylene glycol;
a mixture from padimate 0, octyl salicylate, isopropyl myristate, isopropyl palmitate, octyl methoxycinnamate, laurocapram;
highly disperse silicon dioxide (Aerosil®);
polyoxyethylene-7-glycerol monococoate (Cetiol® HE);
2-octyldodecanol (Eutanol® G);
and mixtures thereof.

The transdermal therapeutic system according to the invention may accordingly be prepared with polyoxyethylene-7-glycerol monococoate (Cetiol® HE) or 2-octyldodecanol (Eutanol® G) as permeation enhancer.

For the transdermal therapeutic system according to the invention, the content of adhesive in the self-adhesive matrix may be from 20 to 90% by weight, preferably from 30 to 80% by weight and especially from 40 to 60% by weight, with the remainder being active ingredient(s), optional permeation enhancer and optional filler, in each case based on the weight of the matrix.

Finally, the problem underlying the invention is solved by a method of producing a transdermal therapeutic system according to the invention, in which method the organic amine(s) and the ACE inhibitor dicarboxylic acid(s) are together incorporated into the matrix solution or suspension and the amine salt(s) is (are) formed in situ in the matrix solution or suspension.

In the method according to the invention of producing a transdermal therapeutic system according to the invention, the amine salt(s) of the ACE inhibitor dicarboxylic acid(s) may be introduced into the matrix directly.

The invention likewise provides for a method of producing a transdermal therapeutic system according to the invention in which the alkali compound(s) and the ACE inhibitor dicarboxylic acid(s) are together incorporated into the matrix solution or suspension and the alkali-compound salt(s) is (are) formed in situ in the matrix solution or suspension.

In the method according to the invention of producing a transdermal therapeutic system according to the invention, the alkali-compound salt(s) of the ACE inhibitor dicarboxylic acid(s) may be introduced into the matrix directly.

In the course of any method according to the invention of producing a transdermal therapeutic system according to the invention, it is also possible for the matrix to be present in the form of a solution or suspension; the solvent or solvents, suspension agent or agents, may optionally be evaporated off in a later process step.

It has therefore now been found, surprisingly, that the salts of ACE inhibitor dicarboxylic acids that are formed by reaction with an organic amine and/or with an alkali compound are largely stable towards decomposition, especially towards intramolecular lactam formation. It has also been found, surprisingly, that the salts of ACE inhibitor dicarboxylic acids that are formed by reaction with a molar equivalent of an organic amine and/or of an alkali compound are largely especially stable towards decomposition, especially towards intramolecular lactam formation. The intramolecular lactam formation is prevented by blockage of the carboxyl group X (see equation) by the amine or the alkali compound, as the case may be.

Since the carboxylate group X is the more acid carboxylate group in the ACE inhibitor dicarboxylic acid, the intramolecular lactam formation can be prevented by as little as a molar equivalent of amine or alkali compound. The salts, especially the mono-salts, of the ACE inhibitor dicarboxylic acids exist in a range that is neutral for the skin, and associated with that they have a minimum potential to irritate the skin. The amine salts or alkali-compound salts of the ACE inhibitor dicarboxylic acids are discharged with a high flux from a matrix former.

Preferably, the pH value established in the application of the transdermal therapeutic system comprising a salt according to the invention, especially a mono-salt, of the ACE inhibitor dicarboxylic acid with an organic amine and/or an alkali compound is from 5.0 to 7.5, preferably from 5.0 to 7.0, and especially preferably from 5.0 to 6.8. That pH value can be determined by way of approximation by shaking the transdermal therapeutic system in a suitable amount of water, for example 25 ml, for a sufficient length of time and, for example at the end of 2 hours, measuring the pH value using a conventional measurement method. Such a pH value range is especially advantageous, since the pH value of the skin is about 4.9 to 5.5.

It has also been found, surprisingly, that a transdermal therapeutic system according to the invention comprising a salt according to the invention of an ACE inhibitor dicarboxylic acid with an organic amine and/or with an alkali compound is especially stable, for example on prolonged storage. In addition, a transdermal therapeutic system according to the invention comprising a salt according to the invention of an ACE inhibitor dicarboxylic acid with an organic amine and/or with an alkali compound is distinguished by a surprisingly high flux (permeation) of the active ingredient during application, which is higher, for example, than that of the free ACE inhibitor dicarboxylic acid. Those effects are especially pronounced in the transdermal therapeutic system according to the invention comprising the salt according to the invention of an ACE inhibitor dicarboxylic acid with an organic amine, especially the mono-salt according to the invention of an ACE inhibitor dicarboxylic acid with an organic amine.

The transdermal therapeutic system according to the invention consists of a top layer (1) impermeable to the active ingredient, one or more self-adhesive layer(s) (2) containing the active ingredient and/or optional permeation enhancers, or one or more matrix layer(s) (4) that are coated with a contact adhesive (5), and a peel-off protective layer (3).

In the transdermal therapeutic system according to the invention, there may be used at least one stable amine salt of an ACE inhibitor dicarboxylic acid, which salt is based on a reaction of an organic amine with an ACE inhibitor dicarboxylic acid. As organic amine there is preferably used a primary aliphatic amine having from 4 to 12 C atoms. Special preference is given to the use of an amine having one amino group (=monoamine). For example, butylamine, pentylamine, hexylamine, heptylamine, octylamine, nonylamine, decylamine, undecylamine, dodecylamine or trometamol (=2-amino-2-hydroxymethyl-1,3-propanediol) may be used. Special preference is given to the use of trometamol as primary aliphatic amine. It is also possible for secondary amines, such as pyrrolidone or derivatives thereof, to be used. Similarly, tertiary amines, such as triethanolamine, can be used.

The transdermal therapeutic system according to the invention may contain, as the active ingredient, ACE inhibitors the active metabolites of which constitute a dicarboxylic acid, such as, for example, imidapril, fosinopril, moexipril, perindopril, spirapril, benazepril, cilazapril, lisinopril, quinapril, enalapril, delapril, ramipril and/or trandolapril. A dicarboxylic acid of an ACE inhibitor is understood to mean the active metabolite of the ACE inhibitor in which the carboxylic acid ester grouping present in the molecule has been hydrolysed. Accordingly, the ACE inhibitor dicarboxylic acid includes, for example, imidaprilate, fosinoprilate, moexiprilate, perindoprilate, spiraprilate, benazeprilate, cilazaprilate, lisinoprilate, quinaprilate, enalaprilate, delaprilate, ramiprilate and/or trandolaprilate. Preferably, the alkali compounds or amine salts of trandolaprilate and/or ramiprolate are used as active ingredient components, the amine salts being preferred.

The amine salt according to the invention is a salt, preferably a mono-salt, in which the molar ratio of ACE inhibitor dicarboxylic acid to amine is less than 1:2, especially from 1:0.5 to 1: <2, preferably from 1:0.5 to 1:1.9. Preference is given to the use of a molar ratio of from 1:0.9 to 1:1.5, especially of 1:1.1 and especially of approximately 1:1.

The stable amine salts of the ACE inhibitor dicarboxylic acids in the matrix solution or suspension can be formed in situ by incorporating the corresponding organic amines and the ACE inhibitor dicarboxylic acids into the matrix together.

The stable amine salts of the ACE inhibitor dicarboxylic acids can, however, also be introduced into the matrix directly.

The amine salts according to the invention of the ACE inhibitor dicarboxylic acids may be dissolved and/or be present in the form of droplets of emulsion in the polymer matrix.

In the transdermal therapeutic system according to the invention there may be used at least one stable alkali-compound salt of an ACE inhibitor dicarboxylic acid, which salt is based on a reaction of an alkali compound with an ACE inhibitor dicarboxylic acid. As alkali compound there are preferably used compounds that include an alkali metal cation. Examples of suitable alkali metal cations are lithium, sodium, potassium, caesium and rubidium cations, of which lithium, sodium and potassium cations are especially preferred. Also preferred as a cation is ammonium (NH₄⁺) (pseudo-alkali metal cation). Preferably, the counter anion of the alkali compound, especially of the alkali metal cation, has proton acceptor properties, and the alkali compound is especially preferably an alkali metal cation-containing salt of a strong or weak, inorganic or organic acid. Preferred examples are alkali compounds such as alkali metal hydroxides, such as, for example, lithium hydroxide, sodium hydroxide or potassium hydroxide, alkali metal alcoholates, such as, for example, lithium methanolate, lithium ethanolate, sodium methanolate, sodium ethanolate, potassium methanolate or potassium ethanolate, or alkali metal carbonates, such as, for example, sodium carbonate or potassium carbonate, or alkali metal hydrogen carbonates, such as, for example, sodium hydrogen carbonate or potassium hydrogen carbonate, or alkali metal tartrates, such as, for example, sodium tartrate or potassium tartrate, or alkali metal maleates, such as, for example, sodium maleate or potassium maleate. Ammonium hydroxide is preferred as ammonium compound. The alkali compound can be used on its own or in admixture with another, suitable, component, or with a solvent.

The transdermal therapeutic system according to the invention may contain as active ingredient one or more of the ACE inhibitors the active metabolites of which constitute a dicarboxylic acid, such as, for example, imidapril, fosinopril, moexipril, perindopril, spirapril, benazepril, cilazapril, lisinopril, quinapril, enalapril, delapril, ramipril and/or trandolapril. A dicarboxylic acid of an ACE inhibitor is understood to mean the active metabolite of the ACE inhibitor in which the carboxylic acid ester grouping present in the molecule has been hydrolysed. Accordingly, the ACE inhibitor dicarboxylic acid includes, for example, imidaprilate, fosinoprilate, moexiprilate, perindoprilate, spiraprilate, benazeprilate, cilazaprilate, lisinoprilate, quinaprilate, enalaprilate, delaprilate, ramiprilate and/or trandolaprilate. Preferably, the alkali-compound salts and/or the amines of trandolaprilate and/or ramiprolate are used as the active ingredient components.

The alkali-compound salt according to the invention is a salt, preferably a mono-salt, in which the molar ratio of ACE inhibitor dicarboxylic acid to alkali compound is less than 1:2, especially from 1:0.5 to 1: <2, further preferably from 1:0.5 to 1:1.9. Preference is given to the use of a molar ratio of from 1:0.9 to 1:1.5, further preferably of 1:1.1 and especially of approximately 1:1.

The stable alkali-compound salts of the ACE inhibitor dicarboxylic acids in the matrix solution or suspension can be formed in situ by incorporating the corresponding alkali compounds and the ACE inhibitor dicarboxylic acids into the matrix solution or suspension together.

The stable alkali-compound salts of the ACE inhibitor dicarboxylic acids can, however, also be introduced into the matrix directly.

The alkali-compound salts according to the invention of the ACE inhibitor dicarboxylic acids may be dissolved and/or be present in the form of droplets of emulsion in the polymer matrix.

Especially preferably, the matrix of the transdermal therapeutic system according to the invention is a non-aqueous matrix, that is to say, a matrix in which the content, or residual content, of water is less than 2% by weight, preferably less than 1% by weight, further preferably less that 0.9% by weight and especially approximately 0.7% by weight or less, based on the weight of the matrix.

The content of ACE inhibitor dicarboxylic acids may be from 2 to 35% by weight, especially from 10 to 25% by weight, based on the weight of the matrix.

For the layer of contact adhesive there may be selected a pressure-sensitive adhesive, for example based on polyurethane, based on polyisobutylene, based on polyvinyl ether, based on polyacrylate, based on silicone, based on styrene block copolymer (for example styrene-isoprene-styrene block copolymer (SIS) or styrene-butadiene-styrene block copolymer) or a mixture thereof. Preference is given to the use of adhesives based on acrylate or polyisobutylene.

The medicinally customary matrix formers, such as polyacrylate, polyisobutylene, silicones, styrene block copolymers (for example styrene-isoprene-styrene block copolymer (SIS)), or a mixture thereof, are used for the matrix. Preference is given to the use of a self-adhesive matrix of polyacrylate which is matrix former and adhesive in one.

The amine salts of the ACE inhibitor dicarboxylic acids are discharged from the matrix former, for example a polyacrylate, with a high flux. In the case of trandolaprilate, a high flux is understood to be from 0.25 to 25 μg/cm²/h, preferably from 0.5 to 5 μg/cm²/h. In the case of ramiprilate, a high flux is understood to be from 0.6 to 63 μg/cm²/h, preferably from 1.2 to 12 μg/cm²/h.

The adhesives based on polyacrylate may be any homopolymer, copolymer or terpolymer containing or consisting of various acrylic acid derivatives.

The polyacrylates may accordingly be polymers of one or more monomers of acrylic acids and other copolymerisable monomers. Furthermore, the polyacrylates may include copolymers of alkyl acrylates and/or alkyl methacrylates and/or copolymerisable secondary monomers or monomers having functional groups. If the amount of the kind being added as monomer is changed, the cohesive properties of the acrylate polymers resulting therefrom may be altered. Generally, the acrylate polymer consists of at least 50% by weight of an acrylate, methacrylate, alkyl acrylate, alkyl methacrylate or acrylamide monomer, from 0 to 20% of a functional monomer, copolymerisable with acrylate, and from 0 to 50% of another monomer.

In the following, various acrylate monomers that can be polymerised on their own or in admixtures are listed, such as, for example, acrylic acid, methacrylic acid, butyl acrylate, butyl methacrylate, hexyl acrylate, hexyl methacrylate, isooctyl acrylate, isooctyl methacrylate, glycidyl methacrylate, 2-hydroxyethyl acrylate, methyl acrylate, methyl methacrylate, 2-ethylhexyl acrylate, 2-ethylhexyl methacrylate, decyl acrylate, decyl methacrylate, dodecyl acrylate, dodecyl methacrylate, tridecyl acrylate and tridecyl methacrylate.

In addition, functional monomers that are copolymerisable with the above-mentioned acrylates, such as, for example, acrylic acid, methacrylic acid, maleic acid, maleic anhydride, hydroxymethyl acrylate, vinyl acetate, hydroxypropyl acrylate, acrylamide, dimethylacryl-amide, tert.-octyl acrylamide, acrylonitrile, dimethylaminoethyl acrylate, dimethylaminoethyl methacrylate, tert.-butylaminoethyl acrylate, tert.-butylaminoethyl methacrylate, methoxyethyl acrylate and methoxyethyl methacrylate, can be used for the copolymerisation.

Further details and examples of pressure-sensitive acrylates that are suitable for the invention are described in Satas Handbook of Pressure Sensitive Adhesive Technology “Acrylic Adhesives”, 3^{rd} edition, (D. Satas, ed.), Van Nostrand Reinhold, New York (1999).

The content of adhesives in the self-adhesive matrix may be from 20 to 90% by weight, from 30 to 80% by weight, especially from 40 to 60% by weight, based on the weight of the matrix.

For the peel-off protective layer there come into consideration polyesters, polyethylene, polypropylene, polysiloxane (for example with a fluorosiliconised coating), polyacrylate, ethylene vinyl acetate, polyurethane, thermoplastic polyurethane, polyisobutene or paper, usually coated with silicone and/or with polyethylene, or a mixture of those substances.

As impermeable top layer there come into consideration sheets of acrylate, acrylonitrile-butadiene-styrene, acrylonitrile (methyl methacrylate) copolymer, acrylonitrile copolymer, ethylene ethyl acrylate, ethylene methyl acrylate, ethylene vinyl acetate, ethylene vinyl acetate copolymer, ethylene vinyl alcohol polymer, ionomers, nylon (polyamide), nylon (polyamide) copolymer, polybutylene, polycarbonate, polyester, polyethylene terephthalate, thermoplastic polyester copolymer, polyethylene copolymer (high density), polyethylene (high molecular weight, high density), polyethylene (intermediate molecular weight, high density), polyethylene (linear low density), polyethylene (low density), polyethylene (medium density), polyethylene oxide, polyimide, polypropylene, polypropylene (coated), polypropylene (oriented), polystyrene, polyurethane, polyvinyl acetate, polyvinyl chloride, polyvinylidene chloride and/or styrene-acrylonitrile, which if required may be metallised or pigmented. For the top layer impermeable to active ingredient, preference is given to polyurethane, ethylene vinyl alcohol polymer and polyester.

As permeation enhancers there may optionally be used saturated and/or unsaturated fatty alcohols each having from 8 to 18 C atoms; tea tree oil; saturated and/or unsaturated cyclic ketones; alkyl methyl sulphoxides; saturated and/or unsaturated fatty acids each having from 8 to 18 C atoms; esters and salts thereof; natural vitamin E; synthetic vitamin E and/or vitamin E derivatives; sorbitan fatty acid esters and ethoxylated sorbitan fatty acid esters; azones (laurocapram); 1-alkylpyrrolidone; block copolymers of polyethylene glycol and dimethylsiloxane having a cationic group at one end; polyoxyethylene-10 stearyl ether; mixtures of polyoxyethylene-10 stearyl ether and glyceryl dilaurate; dodecyl-2-(N,N-dimethylamino)propanoltetradecanoate and/or dodecyl 2-(N,N-dimethylamino)-propionate; N-acetylprolinate esters having >8 C atoms; non-ionic surfactants, for example lauryl ether, esters of polyoxyethylene; dimethyl(arylimino)sulphuran; mixtures of oleic acid analogues and propylene glycol; mixtures from padimate 0, octyl salicylate, isopropyl myristate, isopropyl palmitate, octyl methoxycinnamate, laurocapram; highly disperse silicon dioxide (Aerosil®); polyoxyethylene-7-glycerol monococoate (Cetiol® HE); 2-octyldodecanol (Eutanol® G) or a mixture of different individual components. In the transdermal therapeutic system according to the invention, polyoxyethylene-7-glycerol monococoate (Cetiol® HE) or 2-octyldodecanol (Eutanol® G) is preferred as permeation enhancer.

The following substances, or mixtures thereof, may be used as fillers: metal oxides, such as zinc oxide, magnesium oxide, calcium oxide or titanium oxide, inorganic salts, such as calcium carbonate, magnesium carbonate, sodium carbonate, calcium sulphate, magnesium sulphate or calcium phosphate, clay components, such as talc, kaolin, bentonite or polymer fillers.

There may be used as fillers especially zinc oxide and/or Aerosil.

The transdermal therapeutic system according to the invention comprising a self-adhesive matrix may be constructed as follows. The uppermost layer is the top layer impermeable to the active ingredient. The self-adhesive matrix layer which contains the active ingredient and optional permeation inhibitors and/or fillers then follows. The matrix former is in that case the adhesive. A peel-off protective layer is the final layer.

Alternatively, the transdermal therapeutic system may contain a non-self-adhesive matrix layer that is provided with a separate layer of contact adhesive.

Especially preferably, the transdermal therapeutic system according to the invention comprises an active ingredient-containing matrix layer, which is a non-aqueous matrix layer, that is a matrix layer in which the content, or residual content, of water is less than 2% by weight, preferably less than 1% by weight, further preferably less than 0.9% by weight and especially approximately 0.7% by weight or less, based on the weight of the matrix.

The invention is explained in further detail by way of the Examples which follow, without, however, the scope of the invention being limited thereby.

In the Examples the following components, especially, are used:

As an adhesive based on polyacrylate: Durotak® 78-2353; as an adhesive based on a copolymer of acrylates and vinyl acetate: Durotak® 87-4098; as an adhesive based on styrene-butadiene-styrene block copolymer: Durotak® 87-6173; as an adhesive based on polyisobutylene: Durotak® 87-6430; as a silicone adhesive: BioPSA Hex 7-4302;
As a permeation enhancer: Cetiol® HE: polyoxyethylene-7-glycerol monococoate.

### EXAMPLE 1

Composition of a self-adhesive matrix according to the invention for a TTS comprising a mono-salt of trandolaprilate and trometamol

| Contents | Amount in % by weight |
|---|---|
| Trandolaprilate | 10 |
| Trometamol | 4.5 |
| Cetiol ® HE | 5 |
| Durotak ® 87-2353 | 80.5 |

The percentages by weight relate to the weight of the matrix. The stoichiometric ratio of trandolaprilate to trometamol is 1:1.5.

### Production Process:

In a stirred vessel, trandolaprilate and trometamol are dissolved in a suitable solvent. Subsequently, first Cetiol®HE and then the adhesive (Durotak®) are added and homogenisation is carried out. The resulting mixture is applied to a peel-off film and dried. To the matrix obtained there is then applied a PET film, as top layer impermeable to active ingredient. The TTS is subsequently punched out and packed into sachets.

### EXAMPLE 2

Composition of a self-adhesive matrix according to the invention for a TTS comprising a mono-salt of trandolaprilate and trometamol

| Contents | Amount in % by weight |
|---|---|
| Trandolaprilate | 10 |
| Trometamol | 3 |
| Cetiol ® HE | 5 |
| Durotak ® 87-2353 | 82 |

The percentages by weight relate to the weight of the matrix. The stoichiometric ratio of trandolaprilate to trometamol is 1:1.

Production is carried out analogously to Example 1.

### Stability

Stability of a transdermal therapeutic system comprising a mono-salt of trandolaprilate with trometamol (ratio of trandolaprilate to trometamol is 1:1)

| | | Total of the decomposition |
|---|---|---|
| | | product [%] related to the |
| Storage time | Storage conditions | active ingredient |
| 0 months | Not controlled | 1.0 |
| 3 months | 25° C./60% relative humidity | 0.9 |
| 3 months | 40° C./75% relative humidity | 1.1 |

As can be seen from the Table, a TTS comprising a mono-salt of trandolaprilate exhibits a high degree of stability.

### Skin Irritations:

A transdermal therapeutic system comprising a mono-salt of trandolaprilate with trometamol (ratio of trandolaprilate to trometamol is 1:1.1) exhibits an average value for erythema—an inflammatory reddening of the skin caused by hyperaemia—of 1.

A placebo exhibits an average value for erythema of 1.

According to DRAIZE (Appraisal of the Safety of Chemicals in Food, Drugs and Cosmetics, Association of Food and Drug Officials of the United States, Austin, Tex., 1959), erythemas can be classified as follows:

| Erythema | Value |
|---|---|
| No erythema formation | 0 |
| Very little erythema formation (barely noticeable) | 1 |
| Distinct reddening | 2 |
| Moderate to severe erythema formation | 3 |
| Severe erythema formation (appreciable reddening) or | 4 |
| slough formation (deep lesions) | |

Accordingly, a transdermal therapeutic system comprising a mono-salt of trandolaprilate has a low erythema value, that is, exhibits no skin irritation. There is no difference compared with a placebo.

### pH Value:

To determine the pH value of the transdermal therapeutic systems, 10 cm² portions of laminate were shaken in 25 ml of water for an adequate length of time, that is until a constant pH value was established. The pH value was determined using a conventional pH meter.

| | | Ratio of ACE | pH |
|---|---|---|---|
| ACE inhibitor | Organic amine | inhibitor to amine | value |
| Trandolaprilate | Trometamol | 1:2 | 7.8 |
| Trandolaprilate | Trometamol | 1:1.1 | 6.7 |

### EXAMPLE 3

Composition of a self-adhesive matrix according to the invention for a TTS comprising a salt of ramiprilate and sodium hydroxide (NaOH) in polyacrylate matrix:

| Contents | Amount in % by weight |
|---|---|
| Ramiprilate | 10 |
| NaOH | 1.3 |
| Cetiol ® HE | 5 |
| Durotak ® 87-4098 | 83.7 |

The percentages by weight relate to the weight of the matrix. The stoichiometric ratio of ramiprilate to sodium hydroxide (NaOH) is 1:1.3.

Production is carried out analogously to Example 1.

### EXAMPLE 4

Composition of a self-adhesive matrix according to the invention for a TTS comprising a salt of trandolaprilate and octylamine in silicone matrix

| Contents | Amount in % by weight |
|---|---|
| Trandolaprilate | 10 |
| Octylamine | 4.8 |
| BioPSA Hex 7-4302 | 85.2 |

The percentages by weight relate to the weight of the matrix. The stoichiometric ratio of trandolaprilate to octylamine is 1:1.5.

Production is carried out analogously to Example 1, using a fluorosiliconised peel-off film.

The pH value of the transdermal therapeutic system (determination, see Example 2) is 5.0.

### EXAMPLE 5

Composition of a self-adhesive matrix according to the invention for a TTS comprising a salt of trandolaprilate and dodecylamine in a styrene block copolymer matrix or PIB matrix:

| Contents | Amount in % by weight |
|---|---|
| Trandolaprilate | 10 |
| Dodecylamine | 5.5 |
| Isopropyl palmitate | 5 |
| Durotak ® 87-6173 or | 79.5 |
| Durotak ® 87-6430 | |

The percentages by weight relate to the weight of the matrix. The stoichiometric ratio of trandolaprilate to dodecylamine is 1:1.2.

Production is carried out analogously to Example 1.

### EXAMPLE 6

Composition of a self-adhesive matrix according to the invention for a TTS comprising a salt of trandolaprilate and butylamine in silicone matrix

| Contents | Amount in % by weight |
|---|---|
| Trandolaprilate | 10 |
| Butylamine | 2.7 |
| BioPSA Hex 7-4302 | 87.3 |

The percentages by weight relate to the weight of the matrix. The stoichiometric ratio of trandolaprilate to butylamine is 1:1.5.

Production is carried out analogously to Example 1, using a fluorosiliconised peel-off film.

1. Salt of an ACE inhibitor dicarboxylic acid with at least one organic amine or at least one alkali compound.

2. Salt according to claim 1 with a monoamine as organic amine.

3. Salt according to claim 1 with a primary amine, a secondary amine or a tertiary amine as organic amine.

4. Salt according to claim 3 with an aliphatic primary C₄₋₁₂amine.

5. Salt according to claim 4 with butylamine, pentylamine, hexylamine, heptylamine, octylamine, nonylamine, decylamine, undecylamine, dodecylamine, or trometamol (2-amino-2-hydroxymethyl-1,3-propanediol) as aliphatic primary C₄₋₁₂amine.

6. Salt according to claim 3 with pyrrolidone or a derivative thereof as secondary amine.

7. Salt according to claim 3 with triethanolamine as tertiary amine.

8. Salt according to claim 1, wherein the alkali compound includes an alkali metal cation.

9. Salt according to claim 8, wherein the alkali metal cation is a lithium, sodium or potassium cation.

10. Salt according to claim 1, wherein the ACE inhibitor dicarboxylic acid is selected from the group of the dicarboxylic acids of imidapril, fosinopril, moexipril, perindopril, spirapril, benazepril, cilazapril, lisinopril, quinapril, enalapril, delapril, ramipril and trandolapril.

11. Salt according to claim 10 with an ACE inhibitor dicarboxylic acid of trandolapril or ramipril.

12. Salt according to claim 1 with a molar ratio of ACE inhibitor dicarboxylic acid: organic amine or ACE inhibitor dicarboxylic acid: alkali compound of 1: less than 2.

13. Salt according to claim 12 with a molar ratio of ACE inhibitor dicarboxylic acid: organic amine or ACE inhibitor dicarboxylic acid alkali compound of from 1:0.5 to 1: less than 2, from 1:0.5 to 1:1.9, from 1:0.9 to 1:1.5, of 1:1.1, or of approximately 1:1.

14. (canceled)

15. Transdermal therapeutic system comprising as active ingredient at least one salt according to claim 1.

16. Transdermal therapeutic system according to claim 15 with a monoamine as organic amine.

17. Transdermal therapeutic system according to claim 15 with a primary amine, a secondary amine or a tertiary amine as organic amine.

18. Transdermal therapeutic system according to claim 17 with an aliphatic primary C₄₋₁₂amine.

19. Transdermal therapeutic system according to claim 18 with butylamine, pentylamine, hexylamine, heptylamine, octylamine, nonylamine, decylamine, undecylamine, dodecylamine, or trometamol (2-amino-2-hydroxymethyl-1,3-propanediol) as aliphatic primary C₄₋₁₂amine.

20. Transdermal therapeutic system according to claim 17 with pyrrolidone or a derivative thereof as secondary amine.

21. Transdermal therapeutic system according to claim 17 with triethanolamine as tertiary amine.

22. Transdermal therapeutic system according to claim 15 comprising at least one salt of an ACE inhibitor dicarboxylic acid selected from the group of the dicarboxylic acids of imidapril, fosinopril, moexipril, perindopril, spirapril, benazepril, cilazapril, lisinopril, quinapril, enalapril, delapril, ramipril and trandolapril.

23. Transdermal therapeutic system according to claim 22 comprising a salt of an ACE inhibitor dicarboxylic acid of trandolapril or ramipril.

24. Transdermal therapeutic system according to claim 15 with a molar ratio of ACE inhibitor dicarboxylic acid: organic amine or ACE inhibitor dicarboxylic acid: alkali compound of 1: less than 2.

25. Transdermal therapeutic system according to claim 24 with a molar ratio of ACE inhibitor dicarboxylic acid: organic amine or ACE inhibitor dicarboxylic acid: alkali compound of from 1:0.5 to 1: less than 2, from 1:0.5 to 1:1.9, from 1:0.9 to 1:1.5, of 1:1.1, or approximately 1:1.

26. (canceled)

27. Transdermal therapeutic system according to claim 15 with
a top layer impermeable to active ingredient,
one or more active ingredient-containing self-adhesive matrix layers, and
a peel-off protective layer.

28. Transdermal therapeutic system according to claim 15 with
a top layer impermeable to active ingredient,
one or more active ingredient-containing matrix layers with a layer of contact adhesive provided on the application side, and
a peel-off protective layer.

29. Transdermal therapeutic system according to claim 28 with a non-self-adhesive matrix layer and a separate layer of contact adhesive.

30. Transdermal therapeutic system according to claim 27 in which the active ingredient is dissolved or is present in the form of droplets of emulsion in the matrix.

31. Transdermal therapeutic system according to claim 27, wherein the content of ACE inhibitor dicarboxylic acid is from 2 to 35% by weight, based on the weight of the matrix.

32. Transdermal therapeutic system according to claim 31, wherein the content of ACE inhibitor dicarboxylic acid is from 10 to 25% by weight, based on the weight of the matrix.

33. Transdermal therapeutic system according to claim 15 further comprising a pressure-sensitive adhesive based on polyurethane, polyisobutylene, polyvinyl ether, polyacrylate, silicone, styrene block copolymer or a mixture thereof.

34. Transdermal therapeutic system according to claim 33 with a pressure-sensitive adhesive based on styrene-isoprene-styrene block copolymer (SIS) or styrene-butadiene-styrene block copolymer.

35. (canceled)

36. Transdermal therapeutic system according to claim 15 further comprising a matrix former selected from the group of polyacrylate, polyisobutylene, silicone, styrene block copolymer or a mixture thereof.

37. Transdermal therapeutic system according to claim 36 with a styrene-isoprene-styrene block copolymer (SIS) as matrix former.

38. Transdermal therapeutic system according to claim 36 further comprising a self-adhesive matrix based on polyacrylate.

39. Transdermal therapeutic system according to claim 15 further comprising a contact adhesive or a matrix based on polyacrylate, which may be a homopolymer, copolymer or terpolymer.

40. Transdermal therapeutic system according to claim 39, wherein the polyacrylate comprises one or more acrylic acid derivatives.

41. Transdermal therapeutic system according to claim 39, wherein the polyacrylate consists of acrylate polymer of
at least 50% by weight of an acrylate, methacrylate, alkyl acrylate, alkyl methacrylate or acrylamide monomer,
from 0 to 20% by weight of a functional monomer, copolymerisable with acrylate, and
from 0 to 50% by weight of another monomer.

42. Transdermal therapeutic system according to claim 15 further comprising a permeation enhancer selected from the group formed by
saturated or unsaturated fatty alcohols each having from 8 to 18 C atoms;
tea tree oil;
saturated or unsaturated cyclic ketones;
alkyl methyl sulphoxides;
saturated or unsaturated fatty acids each having from 8 to 18 C atoms;
esters of saturated or unsaturated fatty acids each having from 8 to 18 C atoms;
salts of saturated or unsaturated fatty acids each having from 8 to 18 C atoms;
natural vitamin E;
synthetic vitamin E or vitamin E derivatives;
sorbitan fatty acid esters;
ethoxylated sorbitan fatty acid esters;
azones;
1-alkylpyrrolidone;
block copolymers of polyethylene glycol and dimethylsiloxane having a cationic group at one end;
polyoxyethylene-10 stearyl ether;
a mixture of polyoxyethylene-10 stearyl ether and glyceryl dilaurate;
dodecyl-2-(N,N-dimethylamino)-propanoltetradecanoate or dodecyl 2-(N,N-dimethylamino)-propionate;
N-acetylprolinate esters (N-acetyl-pyrrolidone-2-carboxylic acid esters) having >8 C atoms;
non-ionic surfactants;
esters of polyoxyethylene;
dimethyl(arylimino)sulphuran;
a mixture of oleic acid analogues and propylene glycol;
a mixture from padimate 0, octyl salicylate, isopropyl myristate, isopropyl palmitate, octyl methoxycinnamate, laurocapram;
highly disperse silicon dioxide;
polyoxyethylene-7-glycerol monococoate;
2-octyldodecanol;
and mixtures thereof.

43. (canceled)

44. Transdermal therapeutic system according to claim 27, wherein the content of the adhesive in the self-adhesive matrix is from 20 to 90% by weight, from 30 to 80% by weight from 40 to 60% by weight, based on the weight of the matrix.

45. Method of producing a transdermal therapeutic system according to claim 15, in which the at least one organic amine and the ACE inhibitor dicarboxylic acid are together incorporated into a matrix solution or suspension and the amine salt is formed in situ in the matrix solution or suspension.

46. Method of producing a transdermal therapeutic system according to claim 15 in which the amine salt formed from the at least one organic amine and the ACE inhibitor dicarboxylic acid is introduced into a matrix directly.

47. Method of producing a transdermal therapeutic system according to claim 15 in which the at least one alkali compound and the ACE inhibitor dicarboxylic acid are together incorporated into a matrix solution or suspension and the alkali-compound salt is formed in situ in the matrix solution or suspension.

48. Method of producing a transdermal therapeutic system according to claim 15 in which the alkali-compound salt formed from the at least one alkali compound and the ACE inhibitor dicarboxylic acid is introduced into a matrix directly.

49. Salt of an ACE inhibitor dicarboxylic acid with at least one organic amine and at least one alkali compound.

50. Salt according to claim 49 with a monoamine as organic amine.

51. Salt according to claim 49 with a primary amine, a secondary amine or a tertiary amine as organic amine.

52. Salt according to claim 51 with an aliphatic primary C₄₋₁₂amine.

53. Salt according to claim 52 with butylamine, pentylamine, hexylamine, heptylamine, octylamine, nonylamine, decylamine, undecylamine, dodecylamine, or trometamol (2-amino-2-hydroxymethyl-1,3-propanediol) as aliphatic primary C₄₋₁₂amine.

54. Salt according to claim 51 with pyrrolidone or a derivative thereof as secondary amine.

55. Salt according to claim 51 with triethanolamine as tertiary amine.

56. Salt according to claim 49, wherein the alkali compound includes an alkali metal cation.

57. Salt according to claim 56, wherein the alkali metal cation is a lithium, sodium or potassium cation.

58. Salt according to claim 49, wherein the ACE inhibitor dicarboxylic acid is selected from the group of the dicarboxylic acids of imidapril, fosinopril, moexipril, perindopril, spirapril, benazepril, cilazapril, lisinopril, quinapril, enalapril, delapril, ramipril and trandolapril.

59. Salt according to claim 58 with an ACE inhibitor dicarboxylic acid of trandolapril or ramipril.

60. Salt according to claim 49 with a molar ratio of ACE inhibitor dicarboxylic acid: organic amine or ACE inhibitor dicarboxylic acid: alkali compound of 1: less than 2.

61. Salt according to claim 60 with a molar ratio of ACE inhibitor dicarboxylic acid: organic amine or ACE inhibitor dicarboxylic acid: alkali compound of from 1:0.5 to 1: less than 2, from 1:0.5 to 1:1.9, from 1:0.9 to 1:1.5, of 1:1.1, or of approximately 1:1.

62. Transdermal therapeutic system comprising as active ingredient at least one salt according to claim 49.

63. Transdermal therapeutic system according to claim 62 with a monoamine as organic amine.

64. Transdermal therapeutic system according to claim 62 with a primary amine, a secondary amine or a tertiary amine as organic amine.

65. Transdermal therapeutic system according to claim 64 with an aliphatic primary C₄₋₁₂amine.

66. Transdermal therapeutic system according to claim 65 with butylamine, pentylamine, hexylamine, heptylamine, octylamine, nonylamine, decylamine, undecylamine, dodecylamine, or trometamol (2-amino-2-hydroxymethyl-1,3-propanediol) as aliphatic primary C₄₋₁₂amine.

67. Transdermal therapeutic system according to claim 64 with pyrrolidone or a derivative thereof as secondary amine.

68. Transdermal therapeutic system according to 64 with triethanolamine as tertiary amine.

69. Transdermal therapeutic system according to claim 62 comprising at least one salt of an ACE inhibitor dicarboxylic acid selected from the group of the dicarboxylic acids of imidapril, fosinopril, moexipril, perindopril, spirapril, benazepril, cilazapril, lisinopril, quinapril, enalapril, delapril, ramipril and trandolapril.

70. Transdermal therapeutic system according to claim 69 comprising a salt of an ACE inhibitor dicarboxylic acid of trandolapril or ramipril.

71. Transdermal therapeutic system according to claim 62 with a molar ratio of ACE inhibitor dicarboxylic acid: organic amine or ACE inhibitor dicarboxylic acid: alkali compound of 1: less than 2.

72. Transdermal therapeutic system according to claim 71 with a molar ratio of ACE inhibitor dicarboxylic acid: organic amine or ACE inhibitor dicarboxylic acid: alkali compound of from 1:0.5 to 1: less than 2, from 1:0.5 to 1:1.9, from 1:0.9 to 1:1.5, of 1:1.1, or approximately 1:1.

73. Transdermal therapeutic system according to claim 62 with
a top layer impermeable to active ingredient,
one or more active ingredient-containing self-adhesive matrix layers, and
a peel-off protective layer.

74. Transdermal therapeutic system according to claim 62 with
a top layer impermeable to active ingredient,
one or more active ingredient-containing matrix layers with a layer of contact adhesive provided on the application side, and
a peel-off protective layer.

75. Transdermal therapeutic system according to claim 74 with a non-self-adhesive matrix layer and a separate layer of contact adhesive.

76. Transdermal therapeutic system according to claim 73 in which the active ingredient is dissolved or is present in the form of droplets of emulsion in the matrix.

77. Transdermal therapeutic system according to claim 73, wherein the content of ACE inhibitor dicarboxylic acid is from 2 to 35% by weight, based on the weight of the matrix.

78. Transdermal therapeutic system according to claim 77, wherein the content of ACE inhibitor dicarboxylic acid is from 10 to 25% by weight, based on the weight of the matrix.

79. Transdermal therapeutic system according to claim 62 further comprising a pressure-sensitive adhesive based on polyurethane, polyisobutylene, polyvinyl ether, polyacrylate, silicone, styrene block copolymer or a mixture thereof.

80. Transdermal therapeutic system according to claim 79 with a pressure-sensitive adhesive based on styrene-isoprene-styrene block copolymer (SIS) or styrene-butadiene-styrene block copolymer.

81. Transdermal therapeutic system according to claim 62 further comprising a matrix former selected from the group of polyacrylate, polyisobutylene, silicone, styrene block copolymer or a mixture thereof.

82. Transdermal therapeutic system according to claim 81 with a styrene-isoprene-styrene block copolymer (SIS) as matrix former.

83. Transdermal therapeutic system according to claim 81 further comprising a self-adhesive matrix based on polyacrylate.

84. Transdermal therapeutic system according to claim 62 further comprising a contact adhesive or a matrix based on polyacrylate, which may be a homopolymer, copolymer or terpolymer.

85. Transdermal therapeutic system according to claim 84, wherein the polyacrylate comprises one or more acrylic acid derivatives.

86. Transdermal therapeutic system according to claim 84, wherein the polyacrylate consists of acrylate polymer of
at least 50% by weight of an acrylate, methacrylate, alkyl acrylate, alkyl methacrylate or acrylamide monomer,
from 0 to 20% by weight of a functional monomer, copolymerisable with acrylate, and
from 0 to 50% by weight of another monomer.

87. Transdermal therapeutic system according to claim 62 further comprising a permeation enhancer selected from the group formed by
saturated or unsaturated fatty alcohols each having from 8 to 18 C atoms;
tea tree oil;
saturated or unsaturated cyclic ketones;
alkyl methyl sulphoxides;
saturated or unsaturated fatty acids each having from 8 to 18 C atoms;
esters of saturated or unsaturated fatty acids each having from 8 to 18 C atoms;
salts of saturated or unsaturated fatty acids each having from 8 to 18 C atoms;
natural vitamin E;
synthetic vitamin E or vitamin E derivatives;
sorbitan fatty acid esters;
ethoxylated sorbitan fatty acid esters;
azones;
1-alkylpyrrolidone;
block copolymers of polyethylene glycol and dimethylsiloxane having a cationic group at one end;
polyoxyethylene-10 stearyl ether;
a mixture of polyoxyethylene-10 stearyl ether and glyceryl dilaurate;
dodecyl-2-(N,N-dimethylamino)-propanoltetradecanoate or dodecyl 2-(N,N-dimethylamino)-propionate;
N-acetylprolinate esters (N-acetyl-pyrrolidone-2-carboxylic acid esters) having >8 C atoms;
non-ionic surfactants;
esters of polyoxyethylene;
dimethyl(arylimino)sulphuran;
a mixture of oleic acid analogues and propylene glycol;
a mixture from padimate 0, octyl salicylate, isopropyl myristate, isopropyl palmitate, octyl methoxycinnamate, laurocapram;
highly disperse silicon dioxide;
polyoxyethylene-7-glycerol monococoate;
2-octyldodecanol;
and mixtures thereof.

88. Transdermal therapeutic system according to claim 73, wherein the content of the adhesive in the self-adhesive matrix is from 20 to 90% by weight, from 30 to 80% by weight from 40 to 60% by weight, based on the weight of the matrix.

89. Method of producing a transdermal therapeutic system according to claim 62, in which the at least one organic amine and the ACE inhibitor dicarboxylic acid are together incorporated into a matrix solution or suspension and the amine salt is formed in situ in the matrix solution or suspension.

90. Method of producing a transdermal therapeutic system according to claim 62 in which the amine salt formed from the at least one organic amine and the ACE inhibitor dicarboxylic acid is introduced into a matrix directly.

91. Method of producing a transdermal therapeutic system according to claim 62 in which the at least one alkali compound and the ACE inhibitor dicarboxylic acid are together incorporated into a matrix solution or suspension and the alkali-compound salt is formed in situ in the matrix solution or suspension.

92. Method of producing a transdermal therapeutic system according to claim 62 in which the alkali-compound salt formed from the at least one alkali compound and the ACE inhibitor dicarboxylic acid is introduced into a matrix directly.
